(19) United States
(12) Patent Application Publication
(10) Pub. No.: US 2016/0166448 A1
(43) Pub. Date: Jun. 16, 2016

(54) ADHESIVE ABSORPTIVE SKIN PAD

(71) Applicants: Pablo Laje, Wynnewood, PA (US); William Hughes Peranteau, Philadelphia, PA (US)

(72) Inventors: Pablo Laje, Wynnewood, PA (US); William Hughes Peranteau, Philadelphia, PA (US)

(21) Appl. No.: 14/829,417

(22) Filed: Aug. 18, 2015

Related U.S. Application Data
(60) Provisional application No. 62/090,277, filed on Dec. 10, 2014.

(51) Int. Cl. A61F 13/53; A61F 13/15; A61F 13/58

(52) U.S. Cl. CPC A61F 13/53; A61F 13/58; A61F 13/15577; A61F 2013/15048; A61F 2013/530481

(57) ABSTRACT

An adhesive absorptive pad can be adhesively applied on a patient's skin to prevent spillage and pooling of liquid antiseptics used for skin preparation prior to a surgical or aseptic procedure. The adhesive absorptive pad includes an impermeable posterior layer, an absorbent middle layer, and a permeable anterior layer over the absorbent middle layer. The posterior layer has an adhesive material that can adhere to the skin of the patient in a liquid-tight manner provided on its back side, which is the side of the posterior layer opposite the side facing the absorbent middle layer.

### CROSS-REFERENCE TO RELATED APPLICATIONS

This application claims the benefit of the priority of U.S. Provisional Application for Patent No. 62/090,277, filed on Dec. 10, 2014, which is incorporated herein by reference in its entirety.

### BACKGROUND OF THE INVENTION

1. Field of the Invention

The present invention is in the technical field of devices used to absorb liquids. More particularly, the present invention is in the technical field of flexible adhesive pads used to absorb liquids. More particularly, the present invention is in the technical field of flexible adhesive pads used to absorb liquids that are applied on the skin.

Prior to every surgery or any aseptic procedure, the patient's skin is prepared with liquid antiseptics in order to reduce the risk of a postoperative infection. The skin preparation is done with a combination of isopropyl alcohol and chlorhexidine. Alternatively, iodine-based liquids are used. During the skin preparation, the liquid antiseptic invariably drains away from the area that is being prepared, due to the natural curved surfaces of the body. The spilled liquid antiseptic then accumulates and pools between the operating table and the patient's body. These pools of liquid antiseptic that form beneath the patient are dangerous for two reasons: 1) the prolonged exposure of the patient's skin to the antiseptic causes burns (see References 1-5), and 2) the antiseptics are flammable and therefore are a major hazard for surgical fires (see References 6-7). The use of liquid antiseptics without a barrier to prevent spillage and is an unsafe practice.

2. Related Art

Listed below are some examples of absorbent or spill/leakage protection products.

1. Paper Towel

Material: These are made of paper, which is largely cellulose fiber.

Description: These towels have one or two layers of paper or paper-like material. This is currently the chosen method to prevent spillage of the liquid antiseptics and pooling beneath the patient. The original use of paper towels is to dry the hands of the nurses and surgeons prior to putting on the gown and gloves.

Disadvantages:

a—Paper towels do NOT have an adhesive side so cannot stick to the skin in a water-tight manner (this leads to dripping and accumulation of the liquid antiseptics beneath the patient).

b—Paper towels do NOT maintain integrity after becoming wet, so it disintegrates easily.

c—Does NOT have a posterior impermeable layer, therefore the liquid antiseptics pass through and get in contact with 1) areas of skin that don't need to be prepared, 2) the sheets beneath the patient and 3) elements used for patient monitoring (e.g. EKG leads, electrocautery grounding pad, etc.).

d—Does NOT have a superabsorbent polymer or an equivalent material that solidifies the liquid antiseptic, so the liquid antiseptic that is absorbed remains in liquid state and can continue to drip while the paper towel is removed.

e—Comes in large sizes, so the liquid antiseptic continues to spread away and becomes in contact with other areas of the patient and the elements used for patient monitoring.

f—Does NOT adapt to the curved surfaces of the body, which leaves multiple gaps through which the liquid antiseptic can spill.

g—Is NOT designed to serve the purpose of avoiding spillage and pooling of liquid antiseptic beneath the patient.

2—Cloth towel

Material: Cotton, Polyester.

Description: It is currently an alternate method to avoid spillage of the liquid antiseptics and pooling beneath the patient. The original uses of the cloth towels are: 1) to serve as surgical drapes (they are sterile and are used to mark the surgical field), and 2) to dry the hands of the nurses and surgeons prior to putting on the gown and gloves.

Disadvantages:

a—Does NOT have an adhesive side so cannot stick to the skin in a water-tight manner (this leads to dripping and accumulation of the liquid antiseptics beneath the patient).

b—Does NOT have a posterior impermeable layer, therefore the liquid antiseptic passes through and gets in contact with 1) areas of skin that don't need to be prepared, 2) the sheets beneath the patient and 3) elements used for patient monitoring (e.g. EKG leads, electrocautery grounding pad, etc.).

c—Does NOT have a superabsorbent polymer or an equivalent material that solidifies the liquid antiseptic, so the liquid antiseptic that is absorbed remains in liquid state and can continue to drip while the cloth towel is removed.

d—Comes in large sizes, so the liquid antiseptic continues to spread away and becomes in contact with other areas of the patient and the elements used for patient monitoring.

e—Does NOT adapt to the curved surfaces of the body, which leaves multiple gaps through which the liquid antiseptic can spill.

f—Is NOT designed to serve the purpose of avoiding spillage and pooling of liquid antiseptic beneath the patient.

3—“Chux”

Material: Impermeable plastic layer posteriorly and absorbent fluffy material anteriorly.

Description: The “chux” are large absorptive pads usually placed underneath the patient to absorb the patient's own fluids. The “chux” was designed to help patients with incontinence. The chux is placed underneath the patient and kept until it becomes saturated with the patient's own fluids. At that time it is replaced. “Chuxs” are used at home and in hospitals. Chuxs are NOT being used to avoid spillage of liquid antiseptics during skin preparation.

Disadvantages:

a—Does NOT have an adhesive side so cannot stick to the skin in a water-tight manner (this leads to dripping and accumulation of the liquid antiseptics beneath the patient).

b—Does NOT have a superabsorbent polymer or an equivalent material that solidifies the liquid antiseptic, so the liquid antiseptic that is absorbed remains in liquid state and can continue to drip while the chux is removed.

c—Comes in large sizes, so the liquid antiseptic continues to spread away and becomes in contact with other areas of the patient and the elements used for patient monitoring.

d—Does NOT adapt to the curved surfaces of the body, which leaves multiple gaps through which the liquid antiseptic can spill.

e—Is NOT designed to serve the purpose of avoiding spillage and pooling of liquid antiseptic beneath the patient.

f—Chuxs are designed to collect the patient's own fluids.

4—“Ultrasorb”

Material: Impermeable layer posteriorly and Absorbent material anteriorly.

Description: These are special “chux”-like absorptive pads usually placed underneath the patient to absorb the patient's own fluids. The chux was designed to help patients with incontinence. The “Ultrasorb” is placed underneath the patient and kept until it becomes saturated with the patient's own fluids. At that time it is replaced. “Ultrasorb” pads are used at home and in hospitals. These pads are supplied by Medline. “Ultrasorb” pads are NOT being used to avoid spillage of liquid antiseptics during skin preparation.

Disadvantages:

a—Does NOT have an adhesive side so cannot stick to the skin in a water-tight manner (this leads to dripping and accumulation of the liquid antiseptics beneath the patient).

b—Comes in large sizes, so the liquid antiseptic continues to spread away and becomes in contact with other areas of the patient and the elements used for patient monitoring.

c—Does NOT adapt to the curved surfaces of the body, which leaves multiple gaps through which the liquid antiseptic can spill.

d—Is NOT designed to serve the purpose of avoiding spillage and pooling of liquid antiseptic beneath the patient.

e—“Ultrasorb” pads are designed to collect the patient's own fluids.

5—“Steri-drapes”

Material: Single polyethylene layer with an adhesive band on one edge.

Description: These drapes are impermeable, have an adhesive band, but have no absorptive features. These are used to delineate the surgical field, but because they are not absorptive, the liquid antiseptic drips away from the surgical field and accumulates beneath the patient. This product is supplied by 3M. “Steri-drapes” are NOT being used to avoid spillage of liquid antiseptics during skin preparation.

Disadvantages:

a—Does NOT have any absorptive capabilities, so the liquid antiseptic drips away, comes in contact with the sheets beneath the patient, pools beneath the patient and becomes in contact with elements used for patient monitoring.

b—Is NOT designed to serve the purpose of avoiding spillage and pooling of liquid antiseptic beneath the patient.

6—“Invisishield”

Material: Single polyethylene layer with an adhesive band on one edge.

Description: These drapes are impermeable, have an adhesive band, but have no absorptive features. These are used to delineate the surgical field, but because they are not absorptive, the liquid antiseptic drips away from the surgical field and accumulates beneath the patient. These are supplied by Medline. “Invisishield” is NOT being used to avoid spillage of liquid antiseptics during skin preparation.

Disadvantages:

a—Does NOT have any absorptive capabilities, so the liquid antiseptic drips away, comes in contact with the sheets beneath the patient, pools beneath the patient and becomes in contact with elements used for patient monitoring.

b—Is NOT designed to serve the purpose of avoiding spillage and pooling of liquid antiseptic beneath the patient.

7—Wound Care Products

Material: Multiple embodiments, styles, and features.

Description: These products (e.g. band aids) are meant to be applied on wounds, and the absorptive layers collect the fluids produced by the patient's body. These products are used for skin care (ulcers, wounds, etc.). Wound care products are NOT being used to avoid spillage of liquid antiseptics during skin preparation.

Disadvantages:

a—Are designed to be applied on wounds and collect the patient's own fluid from open wounds.

b—The absorptive component and the adhesive component are on the same side.

8—Feminine Products

Materials: Multiple embodiments, styles, and features.

Description: These products are meant to be worn throughout the day and absorb the fluids produced by the body. These are products used for feminine hygiene. Feminine Products are NOT being used to avoid spillage of liquid antiseptics during skin preparation.

Disadvantages:

a—Are not designed to surround an area of the body surface to be subjected to surgical or aseptic procedures.

The following United States Patents show further examples of absorbent articles. The following list does not constitute an admission that any of the listed patents are prior art.

U.S. Pat. Nos. 7,941,872 and 8,336,121, incorporated by reference entirely herein, show an absorbent pants shield. The pants shields are not shaped to surround an area of the body that is to be subjected to a surgical or aseptic procedure.

U.S. Pat. No. 8,826,911, incorporated by reference entirely herein, shows a surgical drape with a fenestration that allows it to be torn away from the patient's body after use. The drape does not have an adhesive on essentially the entirety of the back side of the drape to allow it to conform to the patient's body while being adhesively attached to the surface of the patient's body in a liquid tight manner.

U.S. Pat. Nos. 8,048,050, 8,937,212, and 8,535,468, incorporated by reference entirely herein, show absorbent feminine hygiene products. These products are not shaped to surround an area of the body that is to be subjected to a surgical or aseptic procedure. These products do not have an adhesive on essentially the entirety of the back side of the drape to allow it to conform to the patient's body while being adhesively attached to the surface of the patient's body in a liquid tight manner.

U.S. Pat. Nos. 8,029,484, 8,353,884, and 8,506,545, incorporated by reference entirely herein, show absorbent incontinence products. These products are not shaped to surround an area of the body that is to be subjected to a surgical or aseptic procedure. These products do not have an adhesive on essentially the entirety of the back side of the drape to allow it to conform to the patient's body while being adhesively attached to the surface of the patient's body in a liquid tight manner.

U.S. Pat. Nos. 7,518,031, 8,084,665, 7,262,071, and 7,888,547, incorporated by reference entirely herein, show absorbent wound dressing products. These products are not shaped to surround an area of the body that is to be subjected to a surgical or aseptic procedure.

U.S. Pat. Nos. 7,652,188 and 8,372,128, incorporated by reference entirely herein, show adhesive bandages. These products have the adhesive and the absorbent material on the side facing the body to absorb discharges from a wound. Accordingly, these products are not suited for surrounding an area of the body that is to be subjected to a surgical or aseptic procedure.

U.S. Pat. No. 8,075,367, incorporated by reference entirely herein, shows an absorbent pad for an underwire brassiere. This product is not suited for surrounding an area of the body that is to be subjected to a surgical or aseptic procedure.

U.S. Pat. No. 8,888,180, incorporated by reference entirely herein, shows an absorbent child safety seat cover for absorbing urine or other body fluids. This product is not suited for surrounding an area of the body that is to be subjected to a surgical or aseptic procedure.

U.S. Pat. Nos. 6,270,873, 9,072,635, and 9,072,807, incorporated by reference entirely herein, show absorbent pads. These products do not have adhesive on the back side and are therefore not suited for surrounding an area of the body that is to be subjected to a surgical or aseptic procedure.

### SUMMARY OF THE INVENTION

The present invention is a flexible, soft, adhesive absorptive pad that is applied on the patient's skin to prevent spillage and pooling of the liquid antiseptics used for skin preparation prior to surgery and antiseptic procedures. The pad has an impermeable posterior layer with adhesive material that sticks to the skin in a water-tight manner, a middle layer containing (in one embodiment) a superabsorbent polymer or (in an alternate embodiment) a sponge-like material (which absorbs the liquid antiseptic that spills), and an anterior layer of permeable material (which conceals the absorptive material of the middle layer and allows the liquid antiseptic to reach that middle layer).

It is an aspect of the present invention to provide an adhesive absorptive pad for being adhesively applied on a patient's skin to prevent spillage and pooling of liquid antiseptics used for skin preparation prior to a surgical or aseptic procedure.

It is another aspect of the present invention to provide an adhesive absorptive pad comprising:

an impermeable posterior layer having a back side and an adhesive material that can adhere to the skin in a liquid-tight manner on said back side;

an absorbent middle layer including absorptive material that can absorb liquid that spills during the procedure; and

an anterior layer including permeable material that covers said absorptive material of said middle layer and allows the liquid to reach said middle layer.

It is yet another aspect of the present invention to provide an adhesive absorptive pad as described above, wherein the absorptive material is a superabsorbent polymer.

It is yet another aspect of the present invention to provide an adhesive absorptive pad as described above, wherein the absorptive material is a sponge-like material.

It is yet another aspect of the present invention to provide an adhesive absorptive pad as described above, wherein the adhesive absorptive pad is flexible such that the adhesive absorptive pad can conform to at least a portion of the patient's body encompassing the area to be subjected to the surgical or aseptic procedure.

It is yet another aspect of the present invention to provide an adhesive absorptive pad as described above, wherein the adhesive absorptive pad is placed on the edges of the area of skin where the liquid antiseptic needs to be applied such that the adhesive absorptive pad absorbs the liquid that spills away from the area that is being prepared for the surgical or aseptic procedure.

It is yet another aspect of the present invention to provide an adhesive absorptive pad as described above, wherein the adhesive absorptive pad has one or more pockets containing the absorptive material.

It is yet another aspect of the present invention to provide an adhesive absorptive pad as described above, wherein the pad has an overall rectangular shape.

It is yet another aspect of the present invention to provide an adhesive absorptive pad as described above, wherein the pad is thin.

It is yet another aspect of the present invention to provide an adhesive absorptive pad as described above, wherein the pockets are created by joining the anterior layer to the posterior layer around the perimeter of each pocket using a mechanism selected from the group consisting of thermal fusion, pressure adhesives, thermal adhesives and combinations thereof.

It is yet another aspect of the present invention to provide an adhesive absorptive pad as described above, wherein the anterior layer of permeable material is bonded to the posterior layer to form one or more pockets containing the absorptive material of the middle layer.

It is yet another aspect of the present invention to provide an adhesive absorptive pad as described above, further comprising a removable non-adhesive layer covering the adhesive material on the back side of the posterior layer to protect the adhesive material when the absorptive adhesive pad is not in use, and wherein the removable non-adhesive layer can be peeled away to expose the adhesive material for application to the patient's body.

It is yet another aspect of the present invention to provide an adhesive absorptive pad as described above, wherein the posterior layer has a front side opposite the back side, the back side being coated with the adhesive material, wherein the front side of the posterior layer faces the middle layer, and wherein the middle layer is intermediate the front side of the posterior layer and the permeable anterior layer.

It is yet another aspect of the present invention to provide an adhesive absorptive pad as described above, wherein the middle layer is composed of absorptive material, and wherein the middle layer is covered by the permeable anterior layer.

It is yet another aspect of the present invention to provide a method for making an adhesive absorptive pad comprising the steps of:

providing an impermeable posterior layer having a back side and a front side;

coating the back side of the impermeable posterior layer with an adhesive material;

laying a middle layer of an absorptive material over at least a portion of the front side of the impermeable posterior layer; and

laying an anterior permeable layer over the middle layer such that the middle layer is positioned intermediate the anterior permeable layer and the impermeable posterior layer.

It is yet another aspect of the present invention to provide an adhesive absorptive pad as described above, wherein the adhesive absorptive pad is in the form of a rectangular sheet divided into a plurality of elongated parallel strips, each of the plurality of elongated parallel strips being delineated from each adjacent strip by a longitudinal frangible zone that allows the plurality of elongated parallel strips to be separated without compromising their integrity.

It is yet another aspect of the present invention to provide an adhesive absorptive pad as described above, wherein each longitudinal frangible zone is coextensive in length with each adjacent pair of the plurality of elongated parallel strips.

It is yet another aspect of the present invention to provide an adhesive absorptive pad as described above, wherein each of the plurality of elongated parallel strips has a length and is divided into a plurality of units arranged in a row along the length of the each of the plurality of elongated parallel strips, wherein each of the plurality of units in each of the plurality of elongated parallel strips is delineated from each adjacent unit in a corresponding one of the plurality of elongated parallel strips by a transverse frangible zone that allows the plurality of units of each of the plurality of elongated parallel strips to be separated without compromising their integrity.

It is yet another aspect of the present invention to provide an adhesive absorptive pad as described above, wherein each of the plurality of elongated parallel strips has a width and each of the plurality of transverse frangible zones extends over the width of the corresponding one of the plurality of elongated parallel strips.

It is yet another aspect of the present invention to provide an adhesive absorptive pad as described above, wherein each unit comprises one or more pockets, each pocket having a perimeter, each pocket being formed by the permeable anterior layer being bonded to the front side of the impermeable posterior layer over an area extending along the perimeter, the middle layer of absorbent material being positioned within each of the one or more pockets.

It is yet another aspect of the present invention to provide an adhesive absorptive pad as described above, wherein the longitudinal frangible zones and the transverse frangible zones allow strips of adhesive absorptive pad of variable length to be obtained from each sheet of adhesive absorptive pad such that the strips obtained from the sheet can be adhered to a patient's skin to completely surround an area of the patient's body that is to be subjected to the surgical or aseptic procedure.

It is yet another aspect of the present invention to provide an adhesive absorptive pad as described above, wherein the longitudinal frangible zones and the transverse frangible zones extend through both the permeable anterior layer and the impermeable posterior layer.

It is yet another aspect of the present invention to provide an adhesive absorptive pad as described above, further comprising a removable non-adhesive layer covering the adhesive material on the back side of the posterior layer to protect the adhesive material when the absorptive adhesive pad is not in use, wherein the longitudinal frangible zones and the transverse frangible zones extend through the removable non-adhesive layer to allow a corresponding portion of the removable non-adhesive layer to remain in place on the adhesive material on the back side of the impermeable posterior layer of each of the strips obtained from the sheet, and wherein the removable non-adhesive layer can be peeled away to expose the adhesive material for application to the patient's body after a strip or unit is separated from the sheet.

It is yet another aspect of the present invention to provide an adhesive absorptive pad as described above, wherein the longitudinal frangible zones and the transverse frangible zones are formed by a cut line, score line, embossed line, or one or more lines defined by one or more perforations extending through both the permeable anterior layer and the impermeable posterior layer.

It is yet another aspect of the present invention to provide an adhesive absorptive pad as described above, wherein the one or more perforations are in the form of holes, cuts, slots, or slits.

It is yet another aspect of the present invention to provide an adhesive absorptive pad as described above, wherein the longitudinal frangible zones and the transverse frangible zones are formed by one or more lines defined by a series of perforations extending through both the permeable anterior layer and the impermeable posterior layer.

It is yet another aspect of the present invention to provide an adhesive absorptive pad as described above, wherein the longitudinal frangible zones and the transverse frangible zones are formed by a cut line, score line, embossed line, or one or more lines defined by one or more perforations extending through the permeable anterior layer, the impermeable posterior layer, and the removable non-adhesive layer.

It is yet another aspect of the present invention to provide an adhesive absorptive pad as described above, wherein a sheet of the adhesive absorptive pad is provided with a central opening dimensioned and shaped to allow an area of a patient's body that is to be subjected to the surgical or aseptic procedure to be exposed when the sheet of the adhesive absorptive pad is adhesively placed on the patient's body such that the area of the patient's body that is to be subjected to the surgical or aseptic procedure is completely surrounded by a seamless sheet of the adhesive absorptive pad.

It is yet another aspect of the present invention to provide an adhesive absorptive pad as described above, further comprising a removable non-adhesive layer covering the adhesive material on the back side of the posterior layer to protect the adhesive material when the absorptive adhesive pad is not in use, wherein the removable non-adhesive layer can be peeled away to expose the adhesive material for application to the patient's body, and wherein the central opening extends through the removable non-adhesive layer.

It is yet another aspect of the present invention to provide an adhesive absorptive pad as described above, wherein the anterior layer of permeable material is bonded to the posterior layer to form one or more pockets containing the absorptive material of the middle layer.

It is yet another aspect of the present invention to provide an adhesive absorptive pad as described above, wherein each pad is connected in series to other such pads to form a roll or strip, and wherein each pad is delineated from adjacent pads by a transverse frangible zone extending transversely relative to the longitudinal axis of the adhesive absorptive pad such that the transverse frangible zones allow strips of adhesive absorptive pad of variable length to be obtained from each roll or strip of adhesive absorptive pads such that the strips of variable length obtained from the roll or strip can be adhered to a patient's skin to completely surround an area of the patient's body that is to be subjected to the surgical or aseptic procedure.

It is yet another aspect of the present invention to provide an adhesive absorptive pad as described above, wherein each pad comprises one or more pockets, each pocket having a perimeter, each pocket being formed by the permeable anterior layer being bonded to the front side of the impermeable posterior layer over an area extending along the perimeter, the middle layer of absorbent material being positioned within each of the one or more pockets.

It is yet another aspect of the present invention to provide an adhesive absorptive pad as described above, wherein the transverse frangible zones extend through both the permeable anterior layer and the impermeable posterior layer.

It is yet another aspect of the present invention to provide an adhesive absorptive pad as described above, further comprising a removable non-adhesive layer covering the adhesive material on the back side of the posterior layer to protect the adhesive material when the absorptive adhesive pad is not in use, wherein transverse frangible zones extend through the removable non-adhesive layer to allow a corresponding portion of the removable non-adhesive layer to remain in place on the adhesive material on the back side of the impermeable posterior layer of each of the strips of variable length obtained from the roll or strip, and wherein the removable non-adhesive layer can be peeled away to expose the adhesive material for application to the patient's body after a strip of variable length is separated from the roll or strip.

It is yet another aspect of the present invention to provide an adhesive absorptive pad as described above, wherein the transverse frangible zones are formed by a cut line, score line, embossed line, or lines defined by one or more perforations extending through both the permeable anterior layer and the impermeable posterior layer.

It is yet another aspect of the present invention to provide an adhesive absorptive pad as described above, wherein the one or more perforations are in the form of holes, cuts, slots, or slits.

It is yet another aspect of the present invention to provide an adhesive absorptive pad as described above, wherein the transverse frangible zones are formed by one or more lines defined by a series of perforations extending through both the permeable anterior layer and the impermeable posterior layer.

It is yet another aspect of the present invention to provide an adhesive absorptive pad as described above, wherein the transverse frangible zones are formed by a cut line, score line, embossed line, or one or more lines defined by one or more perforations extending through the permeable anterior layer, the impermeable posterior layer, and the removable non-adhesive layer.

It is yet another aspect of the present invention to provide an adhesive absorptive pad as described above, wherein each pad having a central opening is connected in series to other such pads to form a roll or strip, and wherein each pad having a central opening is delineated from adjacent such pads by a transverse frangible zone extending transversely relative to the longitudinal axis of the adhesive absorptive pad such that the transverse frangible zones allow each adhesive absorptive pad to be separated from the roll or strip.

It is yet another aspect of the present invention to provide an adhesive absorptive pad having a central opening as described above, wherein the transverse frangible zones extend through both the permeable anterior layer and the impermeable posterior layer.

It is yet another aspect of the present invention to provide an adhesive absorptive pad having a central opening as described above, further comprising a removable non-adhesive layer covering the adhesive material on the back side of the posterior layer to protect the adhesive material when the absorptive adhesive pad is not in use, wherein transverse frangible zones extend through the removable non-adhesive layer to allow a corresponding portion of the removable non-adhesive layer to remain in place on the adhesive material on the back side of the impermeable posterior layer of each pad obtained from the roll or strip, and wherein the removable non-adhesive layer can be peeled away to expose the adhesive material for application to the patient's body after a pad is separated from the roll or strip.

These and other aspects of the present invention will be further elucidated by the Detailed Description of the Invention provided below and the appended drawings.

### BRIEF DESCRIPTION OF THE DRAWINGS

The present invention may be better understood by referencing the accompanying drawings. When the same element appears on multiple drawings, the same number is used to reference it in all the drawings.

FIG. 1 is an anterior view of the adhesive absorptive pad of the present invention.

FIG. 2 is an oblique view of the adhesive absorptive pad of the present invention.

FIG. 3 is a side view of the adhesive absorptive pad of the present invention.

FIG. 4a is a close-up side view of the adhesive absorptive pad of the present invention.

FIG. 4b is a close-up cross sectional view illustrating the various layers of the adhesive absorptive pad of the present invention.

FIG. 5 is a posterior view of the adhesive absorptive pad of the present invention.

FIG. 6 is a schematic lateral view of the adhesive absorptive pad of the present invention depicting each layer of the composition.

FIG. 7 is an anterior view of the adhesive absorptive pad of the present invention.

FIG. 8 is a side view of the adhesive absorptive pad of the present invention.

FIG. 9 is an anterior view of a sheet formed from multiple adhesive absorptive pads of the present invention showing longitudinal and transverse frangible zones for separating the pads from the sheet.

FIG. 10 is an anterior view of the adhesive absorptive pad of the present invention having a central opening.

FIG. 11 is a cross sectional view showing the arrangement of the layers in the adhesive absorptive pad having a central opening in accordance with the present invention.

FIG. 12 is an anterior view of a strip formed from multiple adhesive absorptive pads each having a central opening in accordance with the present invention.

FIG. 13 is an environmental view showing the application of adhesive absorptive pads in accordance with one embodiment of the present invention.

### DETAILED DESCRIPTION OF THE INVENTION

The following is a detailed description of illustrative embodiments of the present invention. As these embodiments of the present invention are described with reference to the aforementioned drawings, various modifications or adaptations of the methods and or specific structures described may become apparent to those skilled in the art. All such modifications, adaptations, or variations that rely upon the teachings of the present invention, and through which these teachings have advanced the art, are considered to be within the spirit and scope of the present invention. Hence, these descriptions and drawings are not to be considered in a limiting sense as it is understood that the present invention is in no way limited to the embodiments illustrated.

The present invention is a device that prevents spillage of the liquid antiseptics used for skin preparation prior to surgery or aseptic procedures. The present invention consists of a flexible, malleable, soft, absorptive, adhesive pad to be applied on the skin prior to the preparation of the skin for surgery or aseptic procedures. The present invention provides a way to completely avoid spillage of the liquid antiseptics away from the area that needs to be disinfected, preventing the pooling of liquid antiseptics in the dependent areas of the patient's body; specifically, the absorptive skin pad has an adhesive material on the posterior side that allows it to stick to the skin in a water-tight manner; the absorptive pad is placed on the edges of the area of skin where the liquid antiseptic needs to be applied; the pad absorbs the liquid antiseptic that spills away from the area that is being prepared for surgery or aseptic procedures.

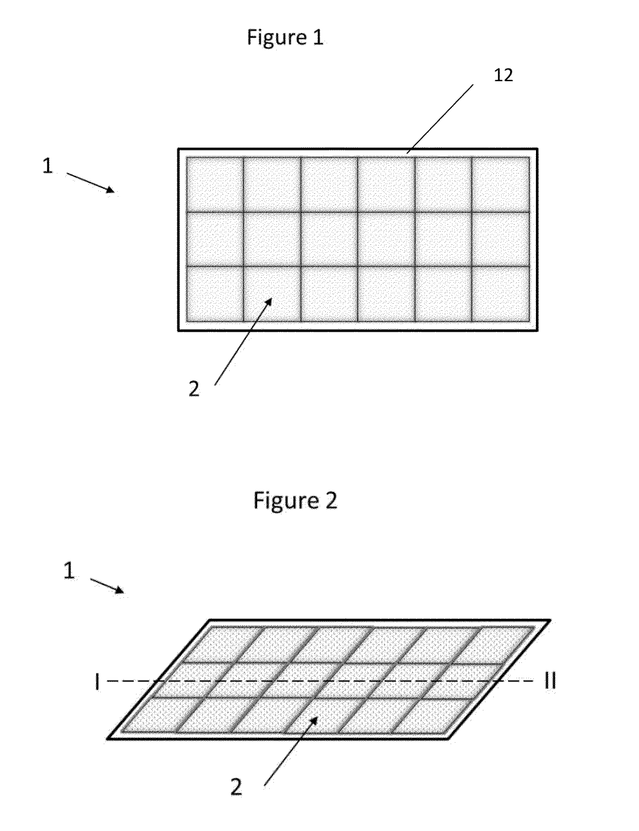

Referring now to the invention in more detail, FIG. 1 shows an anterior view of the adhesive absorptive pad of the present invention 1 which has a variable number of pockets (one to many) containing the absorptive material 2 covered by an anterior permeable layer. The pad has an overall variable rectangular shape.

In further detail, FIG. 2 shows an oblique view of the adhesive absorptive pad 1 with the pockets containing the absorptive material 2, depicting the fact that the pad is thin.

In further detail, FIG. 3 shows a side or lateral view of the adhesive absorptive pad 1 taken at the I-II broken line of FIG. 2. The adhesive absorptive pad 1, in this embodiment, is formed by a posterior adhesive layer 3 of impermeable material, a middle layer of absorptive material 2 and an anterior layer 4 of permeable material. The pockets are created by fusing the anterior layer 4 to the posterior layer 3 by a mechanism such as thermal fusion, pressure adhesives, thermal adhesives or any equivalent method. An example of suitable absorptive material for use in the present invention as the absorptive material 2 is sodium polyacrylate; however, other polymers or other materials that exhibit sufficient absorbance for the types of liquids or liquid mixtures encountered during surgical or aseptic procedures, such as those including body fluids or liquid antiseptics, may also be used.

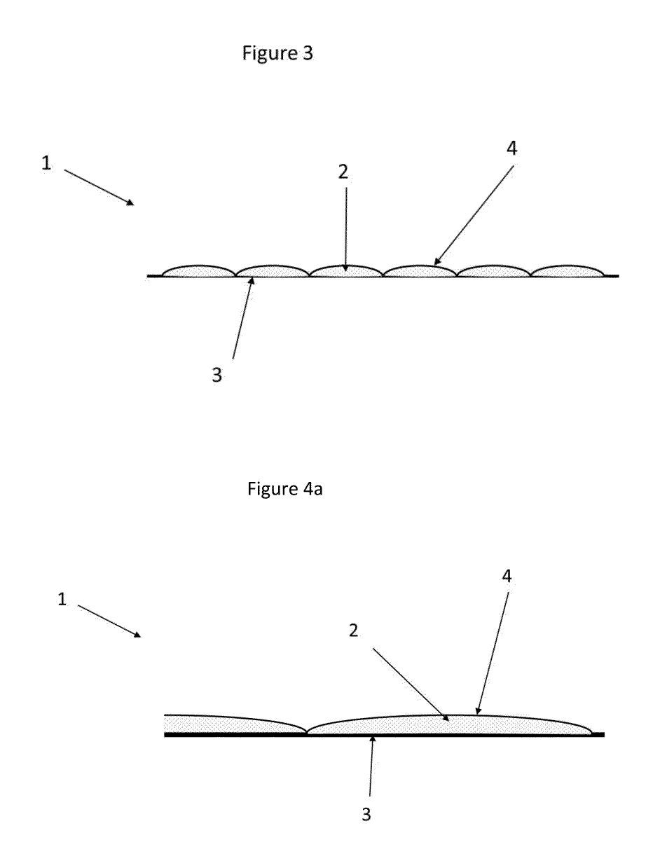

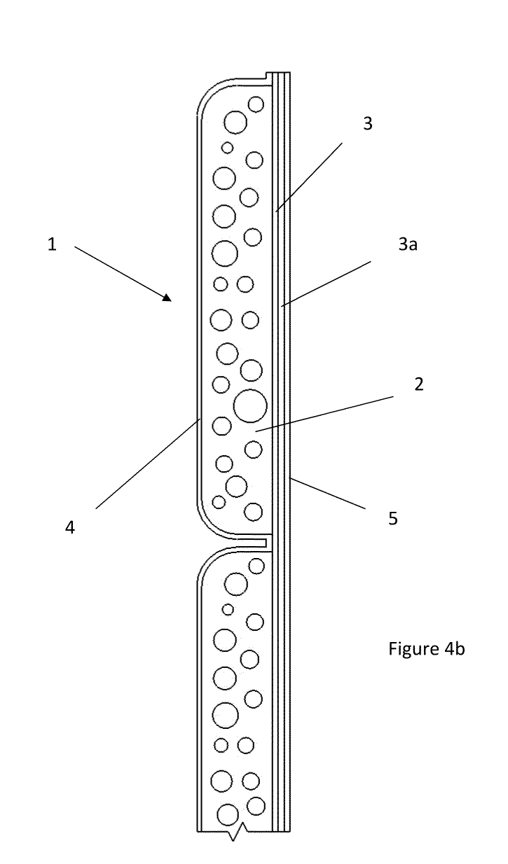

In more detail, FIGS. 4a and 4b show close-up side, or lateral, and cross sectional views of the adhesive absorptive pad 1 which, in this embodiment, is formed by a posterior adhesive layer 3 of impermeable material, a middle layer of absorptive material 2 and an anterior layer 4 of permeable material that is fused to the posterior layer 3 forming pockets where the absorptive material is concealed. The posterior adhesive layer 3 may be inherently adhesive or it may preferably have an adhesive coating 3a applied to its back side.

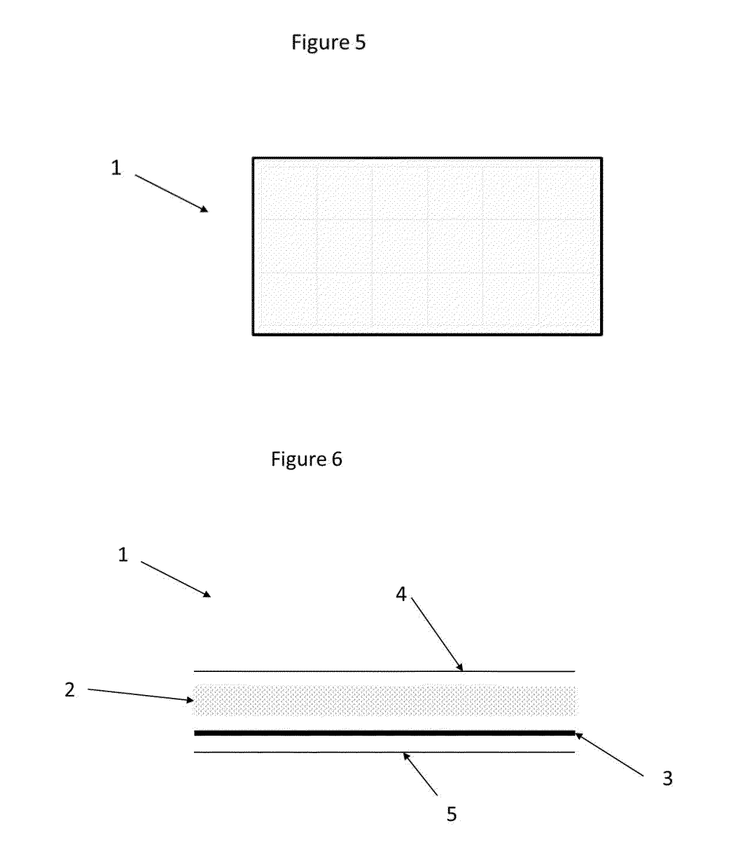

Referring now to FIG. 5, it shows a posterior view of the adhesive absorptive pad 1.

Referring now to FIG. 6, it shows a schematic view of the layers that, in this embodiment, compose the adhesive absorptive pad 1. The adhesive absorptive pad 1 has an impermeable posterior layer 3 coated by an adhesive material that allows it to stick to the skin in a watertight manner. In order to preserve the adhesive material prior to its use, the posterior layer 3 is covered by silicon paper 5 (or any other type of removable non-adhesive material). Above the posterior layer 3 is the middle layer 2 composed of absorptive material, which in turn is covered by the permeable anterior layer 4. The adhesive absorptive pad 1 is created by laying an absorptive material 2 over an impermeable posterior layer 3 (which is coated with an adhesive material on the other side) and laying an anterior permeable layer 4 on the absorptive material 2.

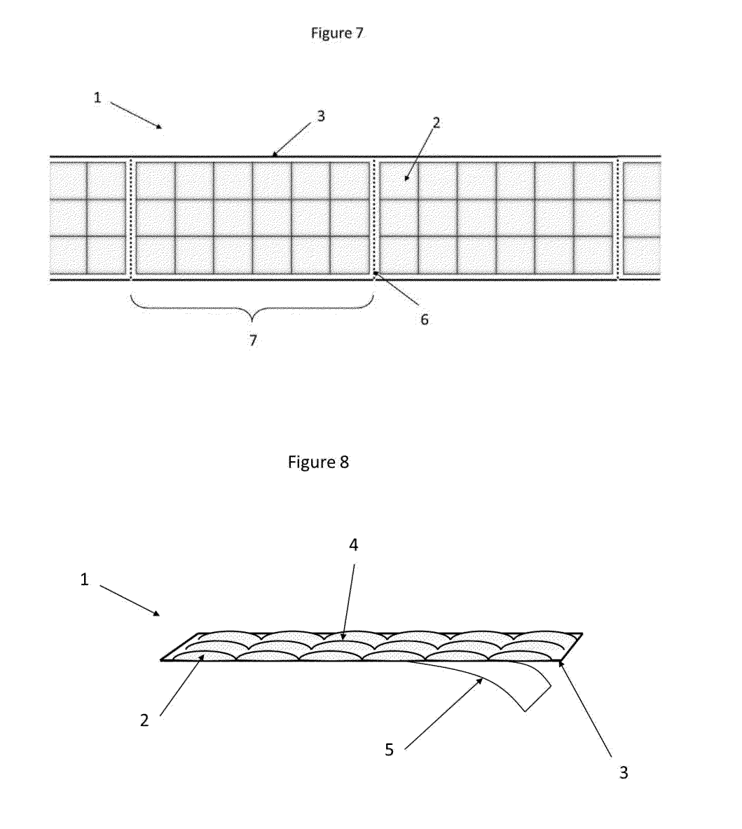

In further detail, FIG. 7 shows that, in this embodiment, the adhesive absorptive pad 1 has individual units 7 linked sequentially to form a strip; the individual units are separated from each other by, in this embodiment, a precut line 6 which allows them to be separated without compromising their integrity. The described configuration of this embodiment allows creating strips of adhesive absorptive pads of variable length that will match the length of the edges of the skin area that needs to be prepared for surgery or aseptic procedures. In an alternate embodiment, the individual units can be in continuity without being separated by a precut area.

In more detail, FIG. 8 is an oblique perspective of the adhesive absorptive pad showing that the posterior layer 3 is covered by a silicon paper 5 (or an equivalent non-adherent material) to preserve the adhesive material before its use. The silicon paper 5 or its equivalent is removed immediately prior to the application of the adhesive absorptive pad on the skin.

Referring now to the present invention of the adhesive absorptive pad, the pad has a variable length and a variable with, such as about 10 centimeters in length by 5 centimeters in width. The overall shape of the adhesive absorptive pad, in this embodiment, is rectangular. The thickness is also variable, such as 1 to 4 millimeters.

The construction details of the present invention, as in the embodiment shown in FIGS. 1 to 8, are that the posterior layer 3 may be made of an impermeable flexible material such as polypropylene or an equivalent. The posterior layer 3 is coated by adhesive tape-like material such as that of the double sided adhesive tapes, which is generally based on acrylics. The adhesive coating is covered and protected by a non-adherent removable layer 5 that may be made of silicone-based paper or an equivalent material. The middle absorptive layer 2 may consist of a superabsorbent polymer such as sodium polyacrylate (or an equivalent product), a foam-like material with high absorptive capacity, or any other type of absorptive material. The anterior layer 4 may be made of any permeable material that is porous enough to allow the liquid antiseptic to penetrate and reach the middle layer 3. The integrity of the material of the anterior layer 4 is such that the absorptive material cannot escape, either prior to or after coming in contact with the liquid antiseptic. The material of the anterior layer 4 is such that it preserves its integrity after it has come in contact with the liquid antiseptic.

In terms of the functionality of the present invention, the adhesive absorptive pads are applied on the skin, at the edges of the skin area where the liquid antiseptic needs to be applied for skin preparation. The adhesive absorptive pads form a polygonal shape delineating the surgical field. An example of the placement of the adhesive absorptive pads 1 around an area of the patient's body to be subjected to a surgical or aseptic procedure, i.e. the surgical field, can be seen in FIG. 13. The liquid antiseptic is applied on the skin area that needs to be prepared, and the excess that spills away from that area is absorbed by the adhesive absorptive pads. Preferably, the adhesive absorptive pad is removed from the patient's body once the skin that has been prepared becomes dry (i.e. once the liquid antiseptic has dried), and prior to placing sterile surgical drapes in preparation for the operation or procedure. In other words, once the liquid antiseptic has dried up at the surgical field, the adhesive absorptive pads are removed and discarded, and the surgical drapes are applied. Preferably, the adhesive absorptive pad is a single-use, disposable product.

The advantages of the present invention, without limitation, include: a water-tight adhesion to the skin; a complete absorption and retention of the excess of liquid antiseptic that is harmful to the patient; a variable length that adapts to all sizes and shapes of surgical fields; a flexible and malleable nature that permits using the adhesive absorptive pads on curved skin surfaces.

In broad embodiment, the present invention is a pad that adheres to the skin and absorbs the liquid antiseptic that spills away from the surgical field during the skin preparation for surgery and aseptic procedures.

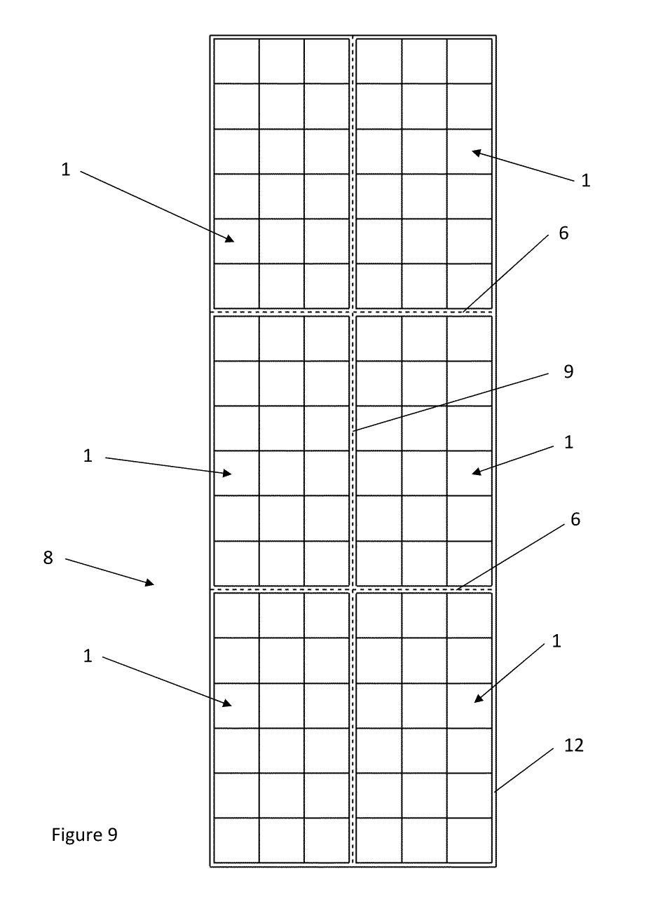

Referring to FIG. 9, yet another embodiment 8 of the present invention can be seen. The embodiment 8 is in the form of a sheet formed from multiple adhesive absorptive pads 1 joined together along the edges thereof by longitudinal frangible zones 9 and transverse frangible zones 6. The frangible zones allow adhesive absorptive strips of varying lengths and widths to be obtained so that the strips are optimally suited for a particular application depending upon the shape and extent of the area of the patient's body that needs to be surrounded by absorptive padding, the area of the skin that needs to be protected from the liquid antiseptics, and the amount of liquid antiseptics that would be expected to run off the site of the procedure.

The pads 1 have straight edges that allow the pads 1 to be placed adjacent to other pads 1 with the edges in abutting contact to as to form a liquid-tight seal. Furthermore, each of the pads 1 has a relatively thin border region 12 along the perimeter of the pad that allows the edges of adjacent pads 1 to be overlapped to further ensure a liquid-tight seal between the pads and around the area of the patient's body to be subjected to a surgical or aseptic procedure. The border regions may be formed by the anterior permeable layer being bonded to the impermeable posterior layer using any suitable bonding technique including those already enumerated herein. Alternatively, the border regions may be formed by the impermeable posterior layer being extended beyond the regions covered by the permeable anterior layer and/or the absorptive middle layer around the perimeter of the pad 1.

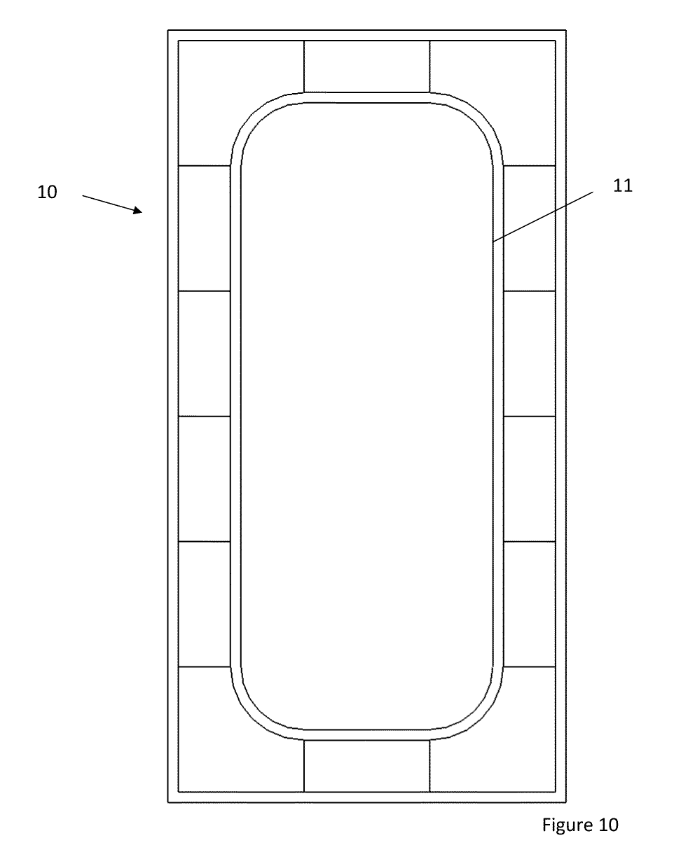

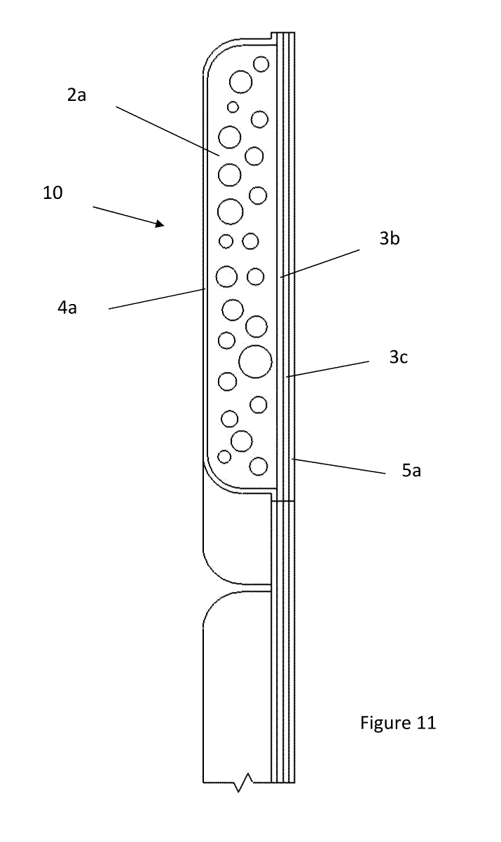

Referring to FIGS. 10 and 11, yet another embodiment 10 of the present invention can be seen. The adhesive absorptive pad 10 differs from the adhesive absorptive pad 1 only in that the adhesive absorptive pad 10 has a central opening 11 extending entirely through the pad that allows a single pad to completely surround the site of the surgical or aseptic procedure without any seams and without the need to use multiple strips. The adhesive absorptive pads 10 can be supplied with central openings 11 that have various sizes and shapes that would be well suited for most commonly encountered surgical and aseptic procedures. FIG. 11 shows the layers in the structure of the adhesive absorptive pad 10, which are identical to those of the adhesive absorptive pad 1 except for differences in shape dictated by the presence of the opening 11. These include the posterior impermeable layer 3b with adhesive coating 3c on its back side, the absorptive middle layer 2a, the permeable anterior layer 4a, and the removable protective liner 5a. The adhesive absorptive pad 10 may also be divided into one or more pockets for the absorptive material of the middle layer as illustrated.

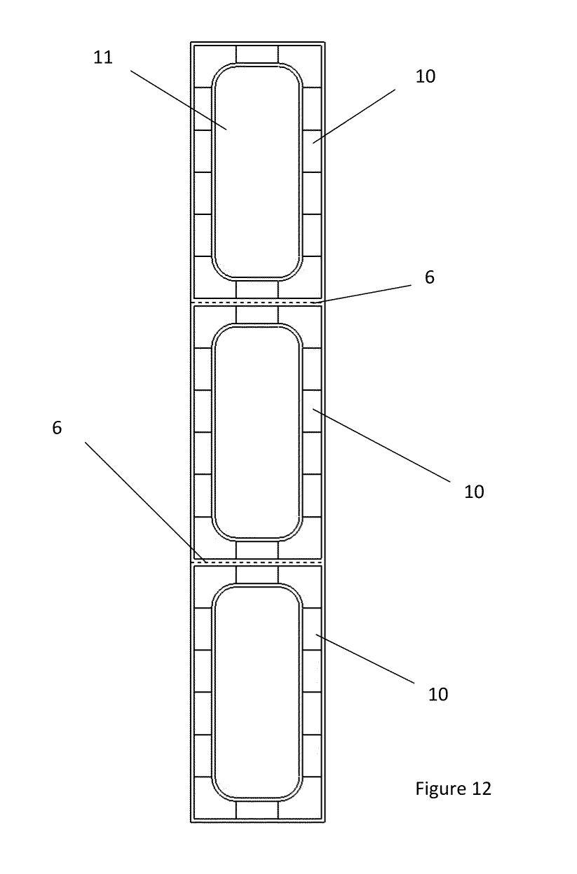

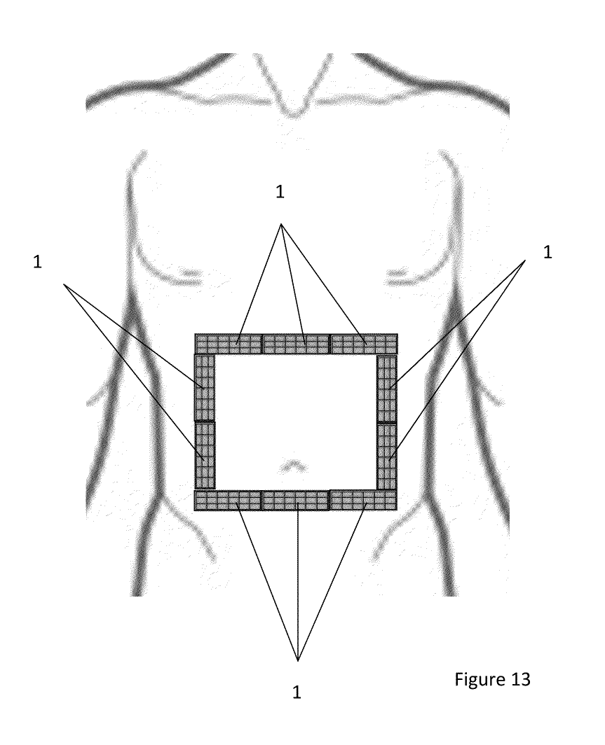

Referring to FIG. 12, multiple adhesive absorptive pads 10 may be combined into strips, or sheets in the same manner as illustrated for the adhesive absorptive pad 1 in FIG. 9, and separated by frangible zones 9 and 6 for convenient dispensing. Furthermore, the adhesive absorptive pads 1 and 10 may be individually wrapped or sheets or strips of these may be individually wrapped, or multiple pads, sheets, strips, or rolls may be provided as a pack. Preferably, individual pads, sheets, strips, or rolls, or packs of these, should be wrapped in such a way as to maintain the products in a sanitary condition.

While the foregoing written description of the present invention enables one of ordinary skill to make and use what is considered presently to be the best model thereof, those of ordinary skill will understand and appreciate the existence of variations, combinations, and equivalents of the specific embodiment, method, and examples herein. The invention should therefore not be limited by the above described embodiment, method and examples, but by all embodiments and methods within the scope and spirit of the invention as claimed.

### REFERENCES

The following references, which are referred to in the Background of the Invention, are incorporated herein by reference in their entirety.
1—Kutsch J, Ottinger D: Neonatal skin and chlorhexidine: a burning experience. Neonatal Netw. 2014; 33(1):19-23.
2—Sanders T H, Hawken S M: Chlorhexidine burns after shoulder arthroscopy. Am J Orthop (Belle Mead N.J.). 2012; 41(4):172-4.
3—Bringué Espuny X, Soria X, Solé E, et al: Chlorhexidine-methanol burns in two extreme preterm newborns. Pediatr Dermatol. 2010; 27(6):676-8.
4—Palmanovich E, Brin Y S, Laver L, et al. Third-degree chemical burns from chlorhexidine local antisepsis. Isr Med Assoc J. 2013; 15(6):323-4.
5—Sivathasan N, Ramamurthy N K, Pabla R S. Chemical burns associated with chlorhexidine-alcohol solution: an avoidable complication? J Burn Care Res. 2010; 31(5):833.
6—Hoshijima H, Takeuchi R, Sato E, et al: Accidental skin burns by fire of an antiseptic agent ignition by the spark of electric cautery. Masui. 2010; 59(11):1438-40.
7—Sivathasan N1, Sivathasan N, Vijayarajan L: Chlorhexidine's complications. J Perioper Pract. 2010; 20(8):300-1.

1. An adhesive absorptive pad for being adhesively applied on a patient's skin to prevent spillage and pooling of liquid antiseptics used for skin preparation prior to a surgical or aseptic procedure, the adhesive absorptive pad comprising:
an impermeable posterior layer having a back side and an adhesive material that can adhere to the skin in a liquid-tight manner on said back side;
an absorbent middle layer including absorptive material that can absorb liquid that spills during the procedure; and
an anterior layer including permeable material that covers said absorptive material of said middle layer and allows the liquid to reach said middle layer.

2. The adhesive absorptive pad according to claim 1, wherein said absorptive material is a superabsorbent polymer.

3. The adhesive absorptive pad according to claim 1, wherein said absorptive material is a sponge-like material.

4. The adhesive absorptive pad according to claim 1, wherein the adhesive absorptive pad is flexible such that the adhesive absorptive pad can conform to at least a portion of the patient's body encompassing the area to be subjected to the procedure.

5. The adhesive absorptive pad according to claim 1, wherein the adhesive absorptive pad has one or more pockets containing the absorptive material, and wherein the adhesive absorptive pad has an overall rectangular shape.

6. The adhesive absorptive pad according to claim 5, wherein the pockets are created by joining the anterior layer to the posterior layer around the perimeter of each pocket using a mechanism selected from the group consisting of thermal fusion, pressure adhesives, thermal adhesives and combinations thereof.

7. The adhesive absorptive pad according to claim 1, wherein the pad is provided with a relatively thin border region along the perimeter of the pad that allows edges of adjacent such pads to be overlapped to further ensure a liquid-tight seal between the pads and around the area of the patient's body to be subjected to a surgical or aseptic procedure.

8. The adhesive absorptive pad according to claim 7, further comprising a removable non-adhesive layer covering said adhesive material on said back side of said posterior layer to protect said adhesive material when the absorptive adhesive pad is not in use, wherein said removable non-adhesive layer can be peeled away to expose said adhesive material for application to the patient's body.

9. The adhesive absorptive pad according to claim 1, wherein said posterior layer has a front side opposite said back side, said back side being coated with said adhesive material, wherein said front side of said posterior layer faces said middle layer, and wherein said middle layer is intermediate said front side of said posterior layer and said permeable anterior layer, and wherein said middle layer is covered by said permeable anterior layer.

10. The adhesive absorptive pad according to claim 9, wherein the adhesive absorptive pad is in the form of a rectangular sheet divided into a plurality of elongated parallel strips, each of said plurality of elongated parallel strips being delineated from each adjacent strip by a longitudinal frangible zone that allows said plurality of elongated parallel strips to be separated without compromising their integrity, and wherein each said longitudinal frangible zone is coextensive in length with each adjacent pair of said plurality of elongated parallel strips.

11. The adhesive absorptive pad according to claim 10, wherein each of said plurality of elongated parallel strips has a length and is divided into a plurality of units arranged in a row along said length of said each of said plurality of elongated parallel strips, wherein each of said plurality of units in each of said plurality of elongated parallel strips is delineated from each adjacent unit in a corresponding one of said plurality of elongated parallel strips by a transverse frangible zone that allows said plurality of units of each of said plurality of elongated parallel strips to be separated without compromising their integrity, and wherein each of said plurality of elongated parallel strips has a width and each of said plurality of transverse frangible zones extends over said width of said corresponding one of said plurality of elongated parallel strips.

12. The adhesive absorptive pad according to claim 11, wherein each said unit comprises one or more pockets, each pocket having a perimeter, each pocket being formed by said permeable anterior layer being bonded to said front side of said impermeable posterior layer over an area extending along said perimeter, said middle layer of absorbent material being positioned within each said one or more pockets, wherein said longitudinal frangible zones and said transverse frangible zones allow strips of adhesive absorptive pad of variable length to be obtained from each sheet of adhesive absorptive pad such that said strips obtained from said sheet can be adhered to a patient's skin to completely surround an area of the patient's body that is to be subjected to the surgical or aseptic procedure, and wherein said longitudinal frangible zones and said transverse frangible zones extend through both said permeable anterior layer and said impermeable posterior layer.

13. The adhesive absorptive pad according to claim 12, further comprising a removable non-adhesive layer covering said adhesive material on said back side of said posterior layer to protect said adhesive material when the absorptive adhesive pad is not in use, wherein said longitudinal frangible zones and said transverse frangible zones extend through said removable non-adhesive layer to allow a corresponding portion of said removable non-adhesive layer to remain in place on said adhesive material on said back side of said impermeable posterior layer of each of said strips obtained from said sheet, and wherein said removable non-adhesive layer can be peeled away to expose said adhesive material for application to the patient's body after a strip or unit is separated from said sheet.

14. The adhesive absorptive pad according to claim 13, wherein said longitudinal frangible zones and said transverse frangible zones are formed by cut lines, score lines, embossed lines, or lines that are each defined by one or more perforations extending through said permeable anterior layer, said impermeable posterior layer, and said removable non-adhesive layer.

15. The adhesive absorptive pad according to claim 14, wherein said one or more perforations are in the form of holes, cuts, slots, or slits.

16. The adhesive absorptive pad according to claim 15, wherein said one or more perforations are a plurality of perforations provided in series.

17. The adhesive absorptive pad according to claim 9, wherein a sheet of the adhesive absorptive pad is provided with a central opening dimensioned and shaped to allow an area of a patient's body that is to be subjected to the surgical or aseptic procedure to be exposed when said sheet of the adhesive absorptive pad is adhesively placed on the patient's body such that the area of the patient's body that is to be subjected to the surgical or aseptic procedure is completely surrounded by a seamless sheet of the adhesive absorptive pad.

18. The adhesive absorptive pad according to claim 17, further comprising a removable non-adhesive layer covering said adhesive material on said back side of said posterior layer to protect said adhesive material when the absorptive adhesive pad is not in use, wherein said removable non-adhesive layer can be peeled away to expose said adhesive material for application to the patient's body, and wherein said central opening extends through said removable non-adhesive layer, and
wherein said anterior layer of permeable material is bonded to the posterior layer to form one or more pockets containing the absorptive material of said middle layer.

19. The adhesive absorptive pad according to claim 9, wherein a plurality of the adhesive absorptive pads are connected end to end to form a strip or a roll of such pads, with each pad being delineated from other adjacent pads by a transverse frangible zone that allows one or more of said plurality of pads to be separated from said strip or roll without compromising the structural integrity of said pads.

20. A method for making an adhesive absorptive pad comprising the steps of:
providing an impermeable posterior layer having a back side and a front side;
coating the back side of the impermeable posterior layer with an adhesive material;
laying a middle layer of an absorptive material over at least a portion of the front side of the impermeable posterior layer; and
laying an anterior permeable layer over the middle layer such that the middle layer is positioned intermediate the anterior permeable layer and the impermeable posterior layer.
